(19) United States
(12) Patent Application Publication
(10) Pub. No.: US 2022/0184312 A1
(43) Pub. Date: Jun. 16, 2022

(54) ASSISTED MANUAL INJECTION DEVICE FOR INJECTING A COMPOSITION CONTAINED IN A CONTAINER

(71) Applicant: EVEON, Montbonnot-Saint-Martin (FR)

(72) Inventors: Arnaud WATTELLIER, Laval (FR); Jean-Claude BEARD, Belley (FR); Christophe DEHAN, Saint-Ismier (FR)

(21) Appl. No.: 17/310,184

(22) PCT Filed: Jan. 23, 2020
(86) PCT No.: PCT/FR2020/050093

§ 371 Date: Jul. 23, 2021

(30) Foreign Application Priority Data
Jan. 24, 2019 (FR) 1900613

(51) Int. Cl. A61M 5/20

(52) U.S. Cl. CPC A61M 5/20; A61B 17/8822

(57) ABSTRACT

An assisted manual injection device for injecting a composition contained in a container, in which the container is provided with a needle at its distal end, and contains a piston, the injection device having a body comprising at least one gripping zone by which the body can be held in a user's hand, a holding element for holding the container configured to receive and hold the container in a stationary manner relative to the body, an electromechanical system comprising a piston rod capable of moving in translation in the container relative to the body of the device to move the piston, an actuation system for actuating the piston rod, a processor capable of communicating with the actuation system, wherein the device comprises at least one force sensor positioned on the body at a gripping zone.

### FIELD OF THE INVENTION

The present invention relates to an assisted manual injection device for injecting a composition contained in a container.

### STATE OF THE ART

Prefilled injection devices are containers currently used for injecting pharmaceutical or cosmetic compositions, and comprise in particular syringes, cartridges and autoinjectors. These containers typically comprise a sealing piston capable of being moved in translation within the container, the container being filled with a pharmaceutical or cosmetic composition in order to provide healthcare workers with ready-to-use injection devices for treating patients.

The container is cylindrical in shape and comprises an open proximal end through which the piston may be inserted in order to sealably close an internal volume of the container enclosing the composition to be injected, a distal end usually provided with a needle through which the composition is injected, and a lateral surface that extends between the proximal end and the distal end of the container. In practice, the piston is capable of being moved following a mechanical effort exerted thereupon by a piston rod movable in translation inside the container, from the proximal end of the container towards the distal end thereof, in order to inject the composition. Such prefilled containers are standard in the medical industry, and are for example prefilled syringes (PFS) with or without needle attached at their distal end, as well as the cartridges. The cartridges further include a septum that will be pierced by a needle at the distal end thereof for the injection.

Compared to empty injection devices that are filled just prior to injection with compositions previously stored in vials, the use of prefilled injection devices has several advantages. In particular, by limiting the preparation steps before the injection, prefilled injection devices are simpler to use, make it possible to improve asepsis, and contribute to reducing dosing errors. Prefilled injection devices encourage and simplify the administration or self-administration of the composition, which reduces the cost of care and facilitates treatment. Finally, prefilled injection devices reduce losses of pharmaceutical or cosmetic composition that may occur when the composition is transferred from a storage vial into a non-prefilled type of injection device. This results in reduced purchase and supply chain costs.

In some cases, the injection of the composition contained in the container with a manual injection device, such as a syringe, can be difficult to achieve because of the effort that must be exerted on the piston rod to expel the composition from the container. This occurs, for example, when a small diameter needle is associated with a specific volume to be injected in an allotted time and certain rheological characteristics of the composition such as high viscosity, and/or when the injection is performed manually by a user who is incapable of pushing hard enough on the piston rod with his fingers.

Thus, for the manual injection of gels, cements, suspensions and other highly viscous products, the operator must exert great force on the piston of the injection syringe in order to ensure passage of the material into the needle. The result:
an injection speed that slows when the diameter of the needle is reduced, for example during injection of gels for filling wrinkles or for articular viscosupplementation,
significant muscular fatigue, even a limitation of the number of actions that can be carried out successively by the same operator,
a reduction in precision, or trembling in carrying out successive actions that the operator must perform when he proceeds with the injection, which may influence the placement, depth or movement of the needle within the injection tissues.

For manual injection devices, manufacturers have added to injection devices gripping surfaces or parts, such as large flanges against which the index and middle finger or thumb can press when the device is held in a tri-dactyl manner, the thumb pressing against the proximal end of the piston rod in order to push said piston rod in the distal direction for injecting the composition. Passive devices have also been added in which the syringe slides.

These surfaces and the gripping parts only resolve the facility of gripping, i.e., the ergonomics, or the mechanical capacity of exerting strong force by increasing the gripping surface. Nevertheless, the problems relative to the maximum force that the hand can exert, muscular fatigue or muscular trembling that result from the muscular contraction of the hand, are unresolved.

Effort-multiplying devices have also been developed.

However, in these effort-multiplying devices, the effort that the user must apply to perform the injection is reduced, but the variation in injection muscular contraction with the resumption of stroke (relaxing the injection means, generally a trigger, that returns to its point of origin, then resuming the pressure) requires a delicate coordination during an injection with progressive movement of the needle, for example the injection of a wrinkle filling gel or a bone cement, and induces a parasitic movement of the hand that can result in possible injury to the patient by the needle.

### DISCLOSURE OF THE INVENTION

A purpose of the invention is therefore to remedy the disadvantages of the prior art.

To that end, the present invention relates to an assisted manual injection device for injecting a composition contained in a container, wherein the container is fitted with a needle at the distal end thereof and contains a piston, the injection device comprising:
a body comprising at least one gripping zone by which the body can be held in a user's hand,
a holding element for holding the container, configured to receive and hold the container in a stationary manner relative to the body,
an electromechanical system comprising:
a piston rod capable of being moved in translation in the container relative to the body of the device in order to move the piston,
an actuation system for actuating the piston rod,
a processor capable of communicating with the actuation system, the device being principally characterized in that it comprises at least one force sensor positioned on the body at a gripping zone, the force sensor being configured to receive, as input, a mechanical effort applied to the sensor by the user during the injection of the composition, and to transmit, to the processor an output signal that depends on the mechanical effort, and the processor is configured to send an instruction to the actuation system depending on the output signal from the sensor in order to actuate the piston rod depending on a predetermined control law, in order to adjust the mechanical effort that needs to be applied by the user to the piston rod in order to inject the composition.

According to other aspects, the injection device has the following different features taken alone or in their technically possible combinations:
the actuation system comprises an effort multiplication system configured to furnish to the piston rod a mechanical effort F2 which is a multiple of the effort F1 exerted by the user on the sensor, and wherein the control law is an effort multiplication law;
the control law defines a movement of the piston rod in a proximal direction along a predetermined distance D1 when the injection is interrupted and the mechanical effort F1 exerted by the user on the sensor is less than a threshold value F1ad determined during a specific duration Δt1;
the piston rod has a starting position, and the control law defines a movement of the piston rod in a proximal direction along a predetermined distance D2 when the injection is terminated and the mechanical effort F1 exerted by the user on the sensor is less than a threshold value F1ad determined during a specific duration Δt2 or when the piston rod is at the distal end of travel;
the needle being positioned in a subcutaneous zone of the human body, the control law defines a movement of the piston rod in a proximal direction according to a predetermined distance D3 in order to perform a calibrated aspiration of a specific volume of a human fluid, in order to detect the possible presence of blood in the needle;
the injection device further comprises a selective activation means allowing the user to selectively activate or deactivate the control of the piston rod by the processor via the control law;
the selective activation means comprises an adjustment means allowing the user to choose a trigger threshold F1s, in such a way that the control of the piston rod by the processor via the control law is triggered only when the effort exerted by the user on the sensor is greater than or equal to the trigger threshold F1s;
the body of the injection device comprises at least two gripping zones on a distal surface of the body of the device, where at least one of the gripping zones is provided with a sensor and is able to receive pressure from the index finger, the middle finger, the ring finger or the little finger of the user, and the piston rod comprises a proximal end comprising a gripping zone, the gripping zone being provided with a sensor and being able to receive the pressure from the user's thumb to allow the device to be held like a syringe;
the gripping zone of the piston rod is positioned on a trigger integral with the piston rod and which extends obliquely or perpendicular to the piston rod from a distal position relative to the proximal end of said piston rod;
the injection device comprises at least three gripping zones arranged on the body of the device around a longitudinal axis of the device, at least one of said gripping zones being provided with a sensor, said gripping zones being able to receive respectively the pressure from the user's index finger, middle finger, or thumb in order to allow the device to be held like a pen;
the holding element of the container comprises a pivotable cover provided with an orifice capable of receiving at least the proximal end of the container, the pivotable cover being capable of pivoting between an injection position in which the orifice is aligned with the travel path of the piston rod, and a free position in which the orifice is offset relative to the travel path of the piston rod and is accessible by the user to allow the insertion of the container into said orifice;
the holding element of the container further comprises a plurality of interchangeable rings provided with orifices of different diameters adapted to receive corresponding containers of different diameters;
the pivotable cover is produced in several parts, of which at least a first part is integral with the body of the device, and at least a second movable part capable of pivoting between the injection position and the free position, the two parts of the pivotable cover thus forming a clamp of variable spacing making it possible to receive and clamp the container in order to maintain it in a stationary position;
the second movable part is capable of being detached from the body of the device;
the second movable part is color-coded to be easily identifiable by the user or is provided with a recognition and electronic reading device in order to specify to the processor the geometric or rheological characteristics of the container and its contents so that the recognition device can automatically calculate the dose of composition delivered for a unitary movement of the piston rod;
the electromechanical system comprises a memory in which control laws and/or threshold values are recorded;
the injection device further comprises a user interface allowing the user to record and select the operating parameters of the device, and to have information feedback from the device about certain operating parameters during the use of the device.

### DESCRIPTION OF THE FIGURES

Other advantages and features of the invention will be seen by reading the following description provided by way of non-limiting illustrative example, with reference to the following appended figures:

FIG. 1 represents a front perspective view of an assisted manual injection device according to one embodiment of the invention, as well as a container containing a composition to be injected, intended to be mounted in a stationary matter on the injection device,

FIG. 2 represents a perspective view of the injection device of FIG. 1, the container being mounted in a stationary manner on the injection device;

FIG. 3 represents a cross-sectional side view of the injection device of FIG. 2;

FIG. 4A represents a cross-sectional side view, centered on an adapter ring allowing a small diameter container to be mounted in a stationary manner on the injection device;

FIG. 4B represents a cross-sectional side view similar to FIG. 4A, in which the adapter ring is removed in order to allow a large diameter container to be mounted on the injection device;

FIG. 5 represents a side view of the injection device according to one embodiment, which shows the holding of the device according to a tridactyl (three-finger) “syringe” type grip;

FIG. 6 represents a side view of the injection device according to one embodiment, which shows the holding of the device according to a pentadactyl (five-finger) “syringe” type grip;

FIG. 7 represents a side view of the injection device according to one embodiment, which shows the holding of the device according to a tridactyl (three-finger) “pen” type grip;

FIG. 8A is a cross-sectional side view of the injection device that shows the step of installing the container on the injection device;

FIG. 8B is a cross-sectional side view of the injection device that shows the step of the piston rod approaching the piston;

FIG. 8C is a cross-sectional side view of the injection device that shows the situation of the injection device in mid-travel of the piston rod;

FIG. 8D is a cross-sectional side view of the injection device that shows the situation of the injection device at end of injection, the piston rod being at end of travel;

FIG. 8E is a cross-sectional side view of the injection device that shows a step of retracting the piston rod at end of injection in order to remove the container from the injection device;

FIG. 9A is a graph showing a first control law example, showing respectively the evolution of the force F2 applied by the actuation system on the piston rod and the speed V of the piston rod as a function of the force F1 applied by the user on the sensor of the injection device;

FIG. 9B is a graph that shows a first embodiment of a second control law example;

FIG. 9C is a graph that shows a second embodiment of a second control law example;

FIG. 9D is a graph that shows a third control law example;

FIG. 9E is a graph that shows a fourth and fifth control law example;

FIG. 10 is a graph that shows the evolution of the axial position of the piston rod according to the axis of injection of the device as a function of time, during an injection of the composition.

### DETAILED DESCRIPTION OF EMBODIMENTS OF THE INVENTION

The invention relates to an assisted manual injection device for injecting a composition contained in a container.

### Container

The container 40, which may also be designated as “primary container,” comprises a body 41 delimited by a lateral surface 42 that extends along a longitudinal axis A between a proximal end 43 and a distal end 44 of the body, and defines a chamber capable of containing a composition to be injected. The container preferably comprises an end 45 to which a needle 46 is attached. It preferably involves a prefilled type container, i.e., filled, sealed with a piston 50, and packaged prior to its subsequent use for injection. Alternatively, the container may initially be empty, then filled and sealed just prior to injection. This last alternative is particularly advantageous when the composition to be injected is obtained by mixing different components, for example during the preparation of a bone cement to be injected. After filling the container with the composition to be injected, the piston 50 is inserted from the proximal end 43 of the container and it separates the composition from the external environment. The container is then mounted in a stationary manner on the injection device of the invention.

The piston is in contact with the composition to be injected, and is configured to be moved by means of the injection device, in a translational movement distally along the longitudinal axis A of the container, in order to inject the composition. This aspect will be explained in more detail in the remainder of the present text.

### Injection Device—General Structure

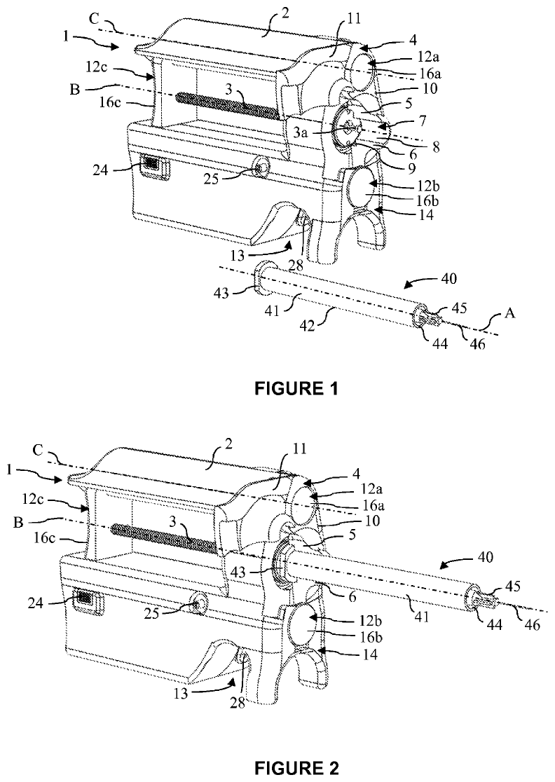

FIGS. 1 and 2 show one embodiment of the injection device 1 according to the invention.

With reference to FIGS. 1 and 2, the injection device 1 comprises a body 2 and a piston rod 3 provided with a head 3a at the distal end thereof, which extends along an axis B that coincides with the axis A of the container 40 when said container is mounted on the body of the device. The piston rod 3 preferably comprises threading functioning as an endless screw.

The device 1 also comprises a system for holding the container, configured to receive and hold the container in a stationary manner relative to the body of the device. The container 1 is removably mounted on the holding system. Thus, the user can change containers for the different injections that must be carried out.

On the device shown in FIG. 1, the holding system comprises a cover 4 at the distal part 14 of the body of the device, said cover delimiting an orifice 7 and being provided with a mounting ring 5 integral with the cover or separable therefrom. The mounting ring 5 forms a protrusion 6 that extends distally from the distal surface 14 of the body, and which extends the orifice 7.

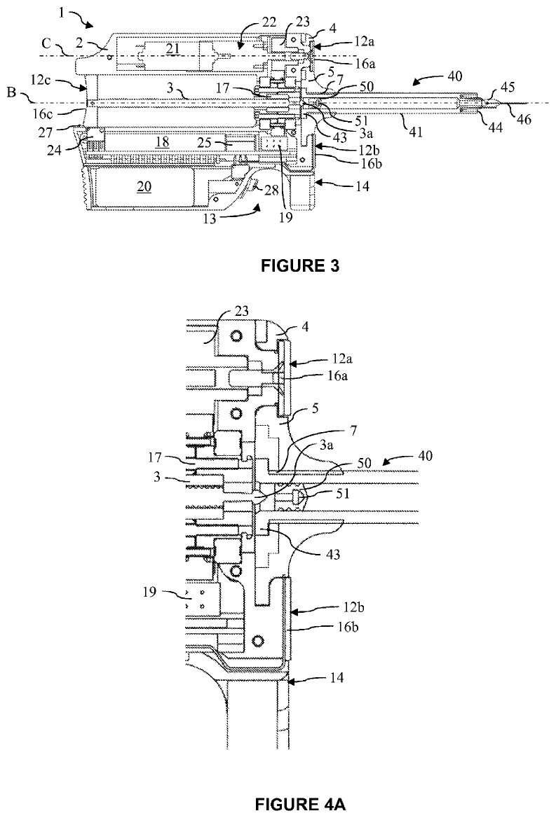

The proximal end 43 of the container is inserted into the orifice 7 the diameter and geometry whereof are adapted for that purpose and for holding the container on the device during its use. For example, as shown in FIG. 3, the orifice may have a first portion 8 the diameter whereof is slightly greater than the diameter of the body of the container, and a second portion 9 which has a shoulder the diameter whereof is greater than that of the first portion of the orifice and which is capable of receiving the flange 43 of the container, such flange typically being present when the container is a syringe as in FIG. 1. Thus, when the syringe is inserted into the orifice and extends through the orifice, the body of the syringe is positioned in the first portion 8 of the orifice and the flange is positioned in the shoulder 9. The syringe is then locked in translation along the axis B.

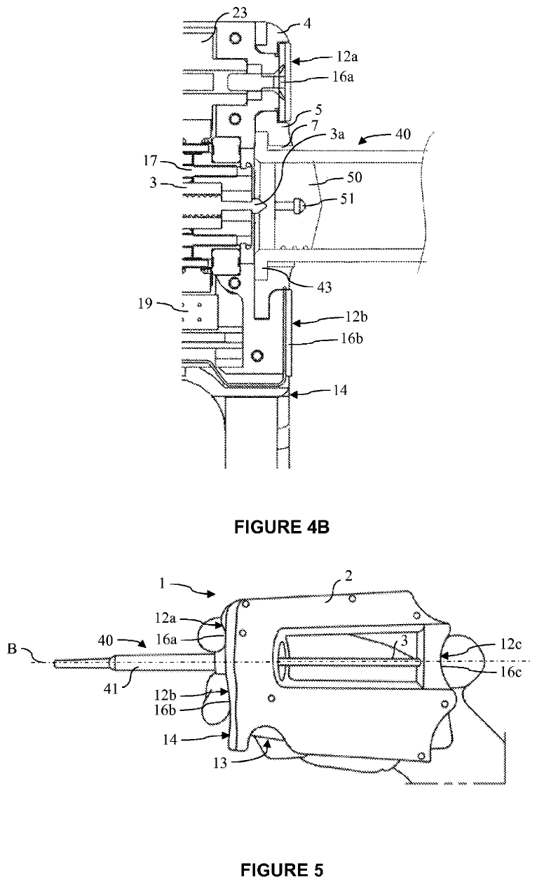

The mounting ring 5 is adjusted based on the dimensions of the containers 40 before being mounted. For example, in FIG. 4A the mounting ring 5 makes it possible to comprise an orifice 7 adapted for inserting a syringe of small diameter. In FIG. 4B, the mounting ring is absent, the orifice being adapted for inserting a large diameter syringe.

According to a preferred embodiment, the cover 4 is pivotable. One end of the cover is pivotably mounted on the body of the device along an axis C parallel to the axis B, in such a way that the cover extends perpendicular to the axis B.

The cover 4 is movable in rotation around the axis C, between an injection position in which the orifice of the ring is aligned with the axis B, and a free position in which the orifice of the ring is offset in relation to the axis B.

In the free position, the orifice 7 is accessible by the user who can then position the container in the orifice by sliding the ring along the orifice. The user then pulls the cover down towards the body of the device to the injection position.

According to an alternative of the preceding embodiment, the pivotable cover 4 is produced in several parts, at least the first part 10 of which is integral with the body of the device, and at least a second part 11 of which is movable in rotation relative to the first part along the axis of rotation C. Both parts of the holding system thus form a clamp of variable spacing, allowing the container to be received and clamped in order to maintain it stationary during the injection.

According to this alternative, the second part 11 of the holding system is interchangeable so as to adapt to containers of different dimensions by simply changing this second part by the user for another one having an orifice of dimensions adapted to the container. This second interchangeable part can also be color-coded to be easily identifiable by the user or provided with a recognition and electronic reading device in order to specify to the device the geometric or rheological characteristics of the container and its contents so that the recognition device can automatically calculate the dose of composition delivered for a unitary movement of the piston rod, as well as other characteristics in order to quickly select the control law.

The body 2 of the injection device is configured to be held in the user's hand.

To that end, the body 2 of the device comprises one or more gripping zones 12a, 12b, 12c where the user can position his fingers in order to hold the device securely by hand, avoiding having it slip out of his hands.

Each gripping zone 12a is preferably in the form of a surface against which one or more of the operator's fingers presses. Alternatively, the gripping zones can comprise a recess 13 wherein the user can insert his finger to make the grip more secure. In this latter case, the user's finger presses against the inner surface of the recess.

With reference to FIGS. 1, 2 and 3, the body of the device comprises two gripping zones 12a, 12b located on the distal surface 14 of the body, and a gripping zone 12c that constitutes the proximal pressure surface of the piston rod 3. The arrangement of these three pressure surfaces according to the embodiment of FIG. 1 defines a “syringe type” handgrip of the device, mimicking the usual three-finger grip of a syringe: thumb, index and middle fingers.

According to the syringe-type grip, the user positions his index finger on the first gripping zone 12a on the distal surface 14 of the body, and positions his middle and possibly ring finger on the second gripping zone 12b. The user positions his thumb on the third gripping zone 12c constituting the pressure zone of the piston rod 3. The index and middle finger are thus placed on either side of the axis B.

The syringe-type grip offers the advantage of being familiar to the user in terms of ergonomics and sensations, whether he or she is a medical professional or a patient. Furthermore, it offers a good view of the movement of the piston rod via the thumb, and therefore of the dose injected during the injection. This allows the user to adjust the injection speed easily by varying the effort exerted by the thumb on the pressure zone of the piston rod and concomitantly on the other fingers in opposite pressure.

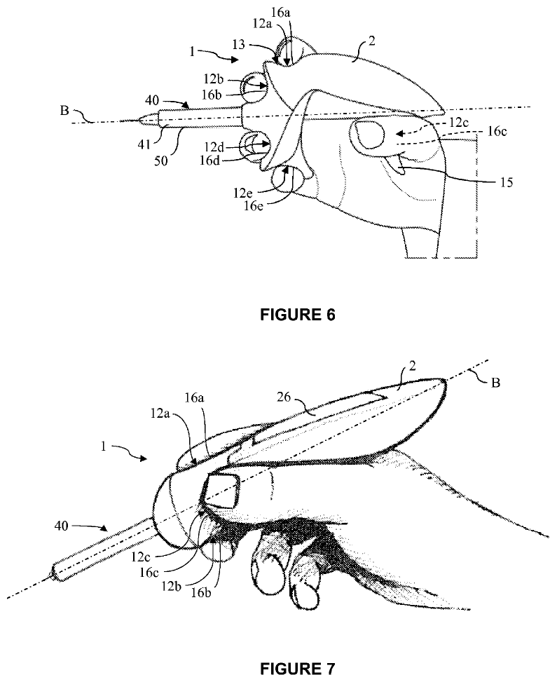

FIGS. 5 and 6 show two embodiments of the injection device 1 according to the invention, enabling a syringe-type grip.

In FIG. 5, the grip is tridactyl in that it involves three of the user's fingers, and is of the syringe type similar to the device in FIGS. 1, 2 and 3. The index finger is positioned on the first gripping zone 12a of the body 2, the middle finger is positioned on the second gripping zone 12b of the body, and the thumb is positioned on the third gripping zone 12c constituting the pressure zone of the piston rod 3.

In FIG. 6, the grip is a variation of the syringe type, and is pentadactyl in that it involves five fingers of the user. The index finger, middle finger, ring finger and little finger are all positioned on respective gripping zones 12a, 12b, 12d, 12e, located on the distal surface 14 of the body. Thus, these four fingers are arranged on either side of the axis B. The thumb is always placed in opposition to the four other fingers, but it is positioned pressed on a trigger 15 integral with the piston rod 3, which extends obliquely or perpendicular from said piston rod. The force applied by the user on the gripping zone 12c of the trigger is therefore applied to the piston rod 3. According to this embodiment the relative gripping zone 12c associated with the thumb is offset with respect to the piston rod 3. This offset makes it possible to reduce the separation between the thumb and the four other fingers with respect to a positioning of the third gripping zone at the end of the piston rod, while still preserving the force applied by the user via the thumb, and concomitantly via the other fingers in opposite pressure, on the piston rod. This results in decreased muscular fatigue of the hand related to holding the hand on the body of the device.

Another possible grip of the injection device 1 according to the invention is the so-called “pen type” grip, in that it mimics the usual handgrip of a writing pen. This grip is shown in FIG. 7.

According to the most common pen-type grip, the user positions the index finger, middle finger and thumb on three respective gripping zones 12a, 12b, 12c located around the body of the device, preferably on a distal portion of said body. The first 12a and second 12b gripping zones, configured to receive the index finger and middle finger, are located substantially along the same axial position along the longitudinal axis of the body. The third gripping zone 12c, configured to receive the thumb, is located proximally with respect to the first 12a and the second 12b gripping zones. The index finger, middle finger and thumb are thus arranged around the axis B.

In FIG. 7, the shown pen-type grip is tridactyl. The ring finger and middle finger are not used.

The pen-type grip offers the advantage of allowing a firm, secure grip of the injection device, since the index finger, middle finger and thumb are all three exerting an effort substantially perpendicular to the longitudinal axis A and oriented towards the body of the device. Furthermore, with this type of grip the hand is closer to the end of the needle, which improves accuracy during the shot, although it does not make it possible to give the movement of the piston rod directly to the user.

However, the grip of the device according to the invention is not limited to the syringe and pen type grips previously described or combinations thereof, so that the device can be produced and gripped differently from these two particular grips.

### Sensors

The device according to the invention comprises at least one force sensor 16a, 16b, 16c which makes it possible to know the force, also called “effort,” exerted by the user to carry out the injection. The force sensor can be static or dynamic.

A static force sensor measures the actual force exerted by the user. It may require calibration in order to calibrate the signal corresponding to a force considered to be zero by the sensor, while the value of the force actually exerted by the user on the sensor may be zero or non-zero.

A dynamic force sensor measures the variations of force. If a force exerted on the sensor is maintained constant over a specific period of time, the sensor considers that there is no more force being applied. It is therefore necessary to carefully choose the time period as a function of the planned usage. One advantage of this type of sensor is that it self-calibrates at rest.

The force sensor 16a, 16b, 16c is configured to receive at the input an effort exerted by the user on said sensor. This sensor, by means of an electromechanical control system, makes it possible to control an actuator according to a control law based on objectives specific to the injection of the composition.

The force sensor 16a, 16b, 16c is positioned in a gripping zone of the body of the injection device. In this way, the force sensor is directly accessible by the user, since he can use his fingers both for gripping the device and for applying pressure to the sensor, and does not need to use other fingers or the other hand to press the sensor. The sensor or sensors are positioned in different gripping zones depending on the type of grip of the device, in order to optimize the ergonomics of the device.

With reference to FIGS. 1, 2 and 3, the injection device 1 comprises up to three sensors 16a, 16b, 16c positioned in such a way as to allow the operation of the device according to the syringe-type grip. Thus, a first sensor 16a is located in the first gripping zone 12a on the distal surface 14 of the body, a second sensor 16b is located in the second gripping zone 12b on the distal surface of the body, and a third sensor 16c is located on the proximal pressure zone 12c of the piston rod 3.

Alternatively, the device may comprise only one or two of the three aforementioned sensors.

According to a preferred embodiment, the device 1 comprises at least the third sensor 16c located in the proximal pressure zone 12c of the piston rod 3. This embodiment offers the advantage that the effort exerted by the user on the sensor is independent of the distribution of the effort exerted on the other fingers at the distal end of the device. The effort exerted by the user on the sensor is directed in the same direction as the movement of the piston rod during the injection, i.e., translation along the axis B in the distal direction. This allows the user to have tactile feedback about the force he is exerting on the sensor as a function of the resultant movement of the piston rod 3 throughout the injection, as well as about the dose injected via the movement of the piston rod, thus improving the precision of the injection.

Quite obviously, the device 1 can comprise only the first sensor 16a and/or the second sensor 16b on the distal surface 14 of the body. In this case, the effort exerted on the sensor is parallel to the movement of the piston rod 3, but oriented in the opposite direction, i.e. in a proximal direction. This embodiment also provides feedback to the operator, although it is less intuitive.

According to the embodiment of FIG. 6, showing the syringe-type pentadactyl grip, the injection device 1 comprises up to five sensors 16a, 16b, 16c, 16d, 16e. Thus, a sensor 16a is located in one of the four gripping zones 12a, 12b, 12d, 12e on the distal surface of the body, and a sensor 16c is located in the fifth gripping zone 12c of the trigger 15.

Again according to FIG. 6, the device may comprise one sensor 16a per gripping zone 12a, for a total of five sensors, or fewer sensors than gripping zones, for example only one, two or three sensors on the distal face 14 of the body of the device and/or one sensor on the trigger 15.

With reference to FIG. 7, the device may comprise up to three sensors 16a, 16b, 16c positioned in such a way as to allow the operation of the device according to the pen-type grip. Thus, a first sensor 16a, a second sensor 16b, and a third sensor 16c are located respectively in the first gripping zone 12a, the second gripping zone 12b and the third gripping zone 12c, on a distal portion of the body of the device.

When the device 1 is designed to function according to the pen-type grip, the presence of the three sensors 16a, 16b, 16c allows the device to receive at the input three efforts of similar intensities directed radially towards the body of the device. This facilitates the use of the device by improving the balance of the body in the user's hand during the injection, which thus greatly improves the precision of the injection.

The injection device 1 further comprises an electromechanical system, said system comprising the piston rod 3, a system 17 for actuating the piston rod, and a processor 18. These different elements are all particularly visible in FIG. 3.

The injection device comprises an ON/OFF switch 19 enabling it to be operated or stopped.

The injection device may comprise an on-board electric energy source 20 for the operation of the electromechanical system, such as a battery or electric cells, thus functioning autonomously. Alternatively, the injection device may be provided with an electrical cable enabling connection to an external source of electrical energy, which allows the device to be made lighter and of smaller size.

The electromechanical system comprises an electric motor 21 and a mechanical reducer 22 allowing the speed of rotation of the motor to be reduced. Such reducer is known per se and conventionally comprises gears 23, which are arranged in proximity to the distal surface 14 of the body of the device of FIG. 3.

The processor 18 is configured to receive at the input a measurement signal from the sensor(s) 16a, i.e., a signal representing a value of force exerted by the user on each sensor, and provides at the output an instruction for the actuation system 17, which is a function of the signal from the sensor.

From the instruction from the processor 18, the actuation system converts the value of the effort measured by the sensor 16a (“input force” or “input effort”) into an effort (or a speed) applied by the piston rod 3 (“output force” or “output effort”) during the use of the device, particularly in order to inject the composition contained in the container 40.

The more sensors 16a, 16b, 16c there are, the greater the amount of input information there is, and the more precisely the output effort is determined from the input effort, by means of the control law.

In particular, the output effort can be a torque. Indeed, according to the embodiment of the device shown in FIGS. 1, 2 and 3, the piston rod 3 comprises threading engaged on threading or a ball nut on the gear shaft, thus functioning as an endless screw. Thus, the torque transmitted to the gearing makes it possible to drive said piston rod in translation, which is rotationally locked. The rotational movement of the gearing is converted into corresponding translational movement of the piston rod along the axis B by the threading or the ball nut of the drive gear.

The instruction sent by the processor 18 to the actuation system 17 is based on a control law programmed and recorded in the processor. The control of the piston rod 3 via the control law may be done by force or by speed. When control is done by speed, the processor 18 receives at the input the force signal from the sensor 16a and provides an output signal to the actuation system 17 in order to control said actuation system in accordance with a certain speed as a function of the input signal.

The processor 18 is advantageously provided with a memory in which control laws and other operating parameters of the device can be recorded.

For example, in order to neutralize the efforts needed for gripping the device without performing an injection, the input force can be corrected from a specific threshold, as well as by linearity, in order to take into account rheological properties of the injected composition and of the needle used, or of the sensitivity desired by the user in particular in the work zone relevant for the usage.

The control law may comprise different operation processes in order to carry out different respective functions.

The injection device 1 preferably comprises a programming connector 24 for connecting the injection device to an electronic apparatus from which the user may configure the injection device. Such an electronic apparatus may for example be a computer, an electronic tablet or a portable phone. By means of the programming connector 24, the user can in particular import control laws into the memory of the device, configure control laws depending on the usage desired, and perform different adjustments of the components of the injection device.

The injection device 1 is advantageously provided with selection means 25, such as an actuable button, making it possible to select one control law from among those that are recorded in the memory, in order for said law to be implemented during the usage of the device.

The injection device 1 is advantageously provided with an adjustment means, such as an actuable button, making it possible to adjust the trigger threshold F1s of the assistance by control law, i.e., the value of the input effort from which the triggering of the selected control law takes place. Thus, as long as the input effort is lower than the trigger threshold F1s, the assistance by control law is not triggered. The value of the input effort corresponds to the value of the output effort. When the input effort becomes greater than or equal to the trigger threshold F1s, the assistance by control law is triggered and assists the user in the use of the device. In order to simplify the use of the device, a unique means 25 can allow both the adjustment of the trigger threshold F1s for assistance and the selection of a control law, as is the case for the device shown in FIGS. 1, 2 and 3.

The injection device 1 can be provided with a user interface, comprising a screen 26 as shown in FIG. 7. This interface allows the user to record and select operating parameters for the device, for example the control laws, the type of container used, or the volume and type of composition to be injected, and to have information feedback from the device, particularly visual feedback via the screen, about said operating parameters during the use of the device. The user can also choose and/or be informed about control laws recorded in the memory, the currently selected control law, the assistance trigger threshold, the battery charge indicator, the volume of composition injected since the device was last turned on, or since the last injection of composition, or since the last selected control law. Other parameters, particularly variable information in a time interval on the order of the duration of an injection (a few seconds to a few minutes) can also be provided to the user via the interface, such as the input effort, the output effort, and the speed of the piston rod, i.e., the injection speed.

### Method of Operation

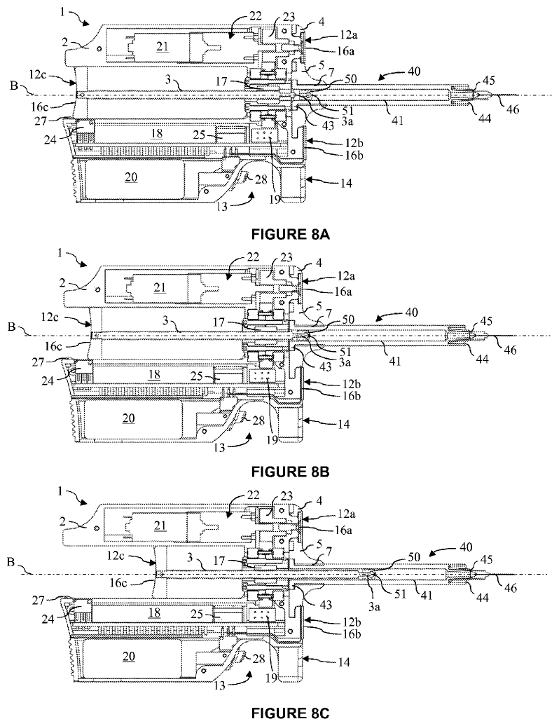

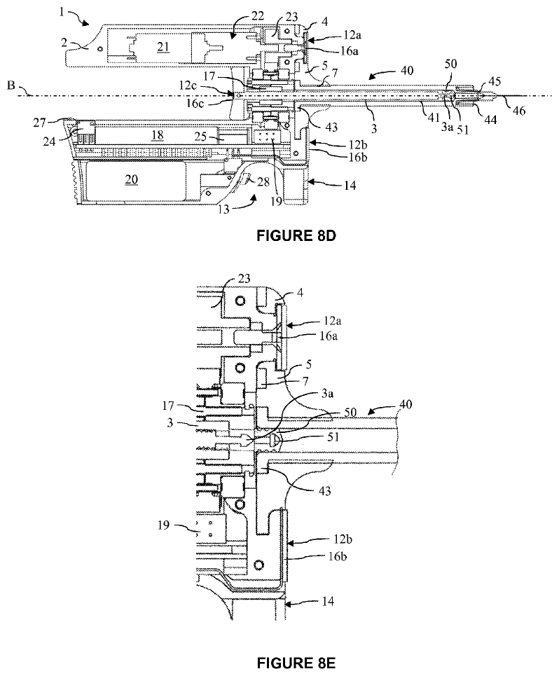

The operation of the injection device 1 will now be described with reference to FIG. 2 and to FIGS. 8A-E to show the injection of a composition contained in a PFS type container 40.

With reference to FIG. 2, the syringe 40 is mounted on the injection device 1. The pivoting cover 4 is opened, in other words the second part 11 of the cover is separated from the first part 10. The syringe 40 is moved perpendicularly to the axis B in such a way as to insert the flange 43 into the shoulder 9 of the mounting ring 5.

The second part 11 of the cover is then joined with the first part 10 in order to clamp the proximal part of the syringe 40 and to maintain said syringe in a stationary manner and in alignment with the travel path of the piston rod. The injection device is then as represented in FIG. 8A.

The gripping zone 12c of the piston rod 3 is in contact with a proximal stop element 27 that limits the proximal end-of-travel of the piston rod. In other words, the piston rod 3 cannot be in a proximal position further than that of FIG. 8A. This position, called “starting position,” is detected by the processor 18.

With reference to FIG. 8B, the electromechanical system preferably launches an automated step of approaching the piston 50 by the piston rod 3. The piston rod is therefore moved distally, automatically without intervention of the user on the piston rod, until it is pressed against the piston 50.

At the end of the approach step, the distal end or head 3a of the piston rod 3 is inserted into the piston 50, the piston being advantageously provided with a recess 51 the shape whereof corresponds to that of the distal end 3a of the piston rod in order to ensure better contact between the piston rod and the piston, which improves the precision of the injection. Moreover, the gripping zone 12c of the piston rod is located at a specific distance from the proximal stop element 27. This distance is detected by the processor. The piston rod is then located in a position called “starting position”, and is ready to be actuated.

The user may then proceed with the injection of the composition by exerting a force on one or more sensors 16a, 16b, 16c of the device. FIG. 8C shows the situation in distal mid-travel of the piston rod.

The piston rod 3 is moved distally in the container 40, driving the piston 50 with it as the composition is injected. FIG. 8D shows the situation at the distal end-of-travel of the piston rod. All the composition has been injected, and the piston 50 is stopped against the distal end of the container.

With reference to FIG. 8E, the electromechanical system preferably launches an automated step of separation from the piston rod 3. The piston rod is therefore moved proximally, automatically without intervention from the user on the piston rod, until the gripping zone 12c of the piston rod comes into contact with the proximal stop element 27.

The piston rod is located outside the container, which container may then be removed from the holding system of the device. The user can then proceed with loading a new container and proceed with another injection, or may turn off the device.

### Control Law Examples

Examples of operating processes corresponding to control laws will now be described according to several embodiments. The embodiments may be combined with each other in such a way that the device may be configured to operate according to one or several of the operating processes described.

### Simple Law

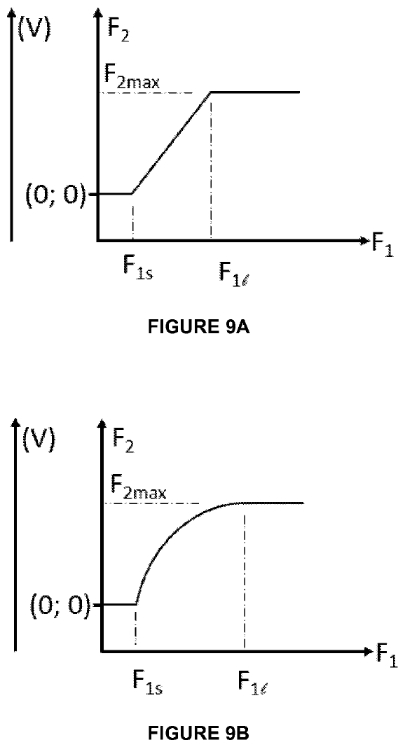

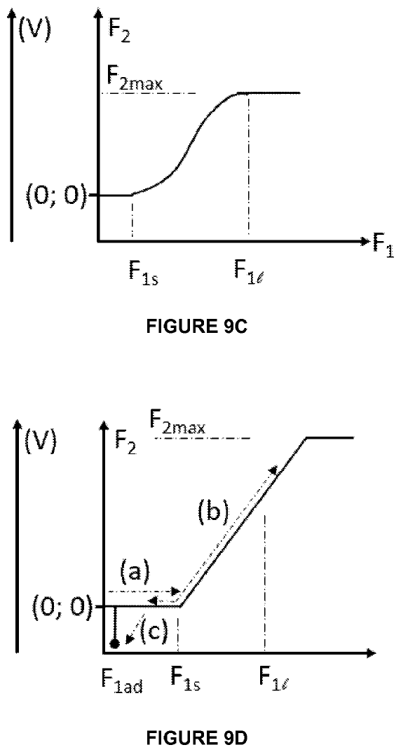

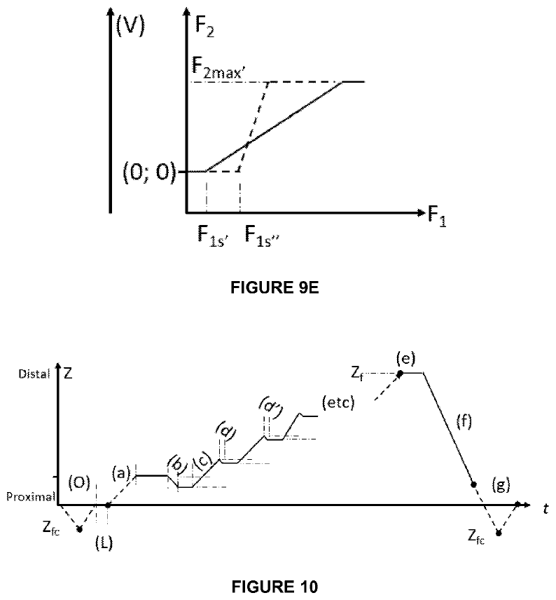

According to a first example shown in FIG. 9A, the control law is a simple injection law according to which the output effort F2 is substantially proportional to the input effort F1.

Since the injection device according to the invention is also speed-controllable, the speed furnished by the actuation system at the output is measured on the y-axis, in parallel to the output effort F2. The observations made with regard to the output effort F2 and the output speed F2 are the same.

The output effort F2 remains zero until the input effort F1 exerted by the user on the sensor(s) reaches the trigger threshold F1s after which assistance by control law is triggered. The output effort F2 then increases with the input effort F1.

In FIG. 9A, the increase of F2 with F1 is represented by a straight line and corresponds to a linear function. This representation is simplified. In practice, the device behavior depends in particular on the type of syringe, the rate of fill of the syringe, and the viscosity of the composition, so that the increase of F2 with F1 is represented rather by a curve.

The output effort F2 increases up to a limit F2max when the input effort reaches F1I. This value F2max is recorded in the memory of the injection device particularly during its manufacture, and makes it possible to avoid any risk of explosion of the syringe or inadvertent ejection of the needle due to excessive output effort F2.

### Effort Multiplication

In a second example, the control law comprises an effort multiplication process. According to this process, the output effort F2 (respectively the output speed) of the actuation system is increased primarily according to a multiple of the value of the input effort F1.

To that end, the actuation system 17 comprises an effort multiplication system configured to furnish an output effort F2 for actuating the piston rod 3 which is a multiple of the value of the effort F1 received at the input by the processor 18.

FIG. 9B shows a first embodiment of an effort multiplication control law. The output effort F2 remains zero until the input effort F1 exerted by the user on the sensor(s) 16a, 16b, 16c reaches the trigger threshold F1s after which assistance by control law is triggered. The output effort F2 then increases very sharply, asymptotically, and is stabilized at F2max when the input effort F1 reaches a limit F1I. The injection is therefore greatly facilitated for low values of F1<F1I. The injection device is very sensitive starting from threshold F1s and not very sensitive after the limit F1I.

FIG. 9C shows a second embodiment of an effort multiplication control law. The output effort F2 remains zero until the input effort F1 exerted by the user on the sensor(s) 16a, 16b, 16c reaches the trigger threshold F1s after which assistance by control law is triggered. The output effort F2 then increases slowly, then strongly until it is stabilized at F2max when the input effort F1 reaches the limit F1I. The triggering of assistance is therefore more progressive than for the first embodiment by offering a wide range of sensitivity within a range of operation.

### Anti-Drip Device

In the injection devices of the state-of-the-art, the internal pressure in the container and the deformation in the actuation system of the piston rod during injection of viscous products results in an injection residue of the composition, even though the operator has ceased pressing on the piston rod of the syringe, by a mechanism of releasing the built-up internal pressure. This surplus of injected composition, which is undesirable, is problematic in a case where a very precise dose of composition is required or the user wishes to quickly move the needle and extraneous material should not be deposited during removal of the needle, as is the case for some bone cements that polymerize quickly, used in vertebroplasty, which should not be deposited outside the treated vertebrae.

In order to overcome this disadvantage, according to a third example, the controller comprises a process that makes it possible to carry out a backwards return of the piston rod 3, in other words a movement of the piston rod in the proximal direction relative to the body 2 of the device according to a specific distance, when the user wishes to stop the injection, either definitively for example when all the composition has been injected and the piston rod 3 is at end-of-travel, or from time to time for example when the operator carries out a series of injections while including a stop time between two consecutive injections.

This process thus makes it possible to eliminate any surplus composition, and thus to inject a precise dose of composition at selected sites, without smears.

When the injection is interrupted, momentarily or definitively, the effort F1 exerted by the user on the sensor 16a, 16b, 16c falls below a specific retraction threshold F1ad, equal to or less than the activation threshold F1s, without becoming zero. The value of the effort F1 of the signal received by the processor 18 from the sensor decreases even though the injection device is in operation. In this case, the processor 18 is configured to send an instruction to the actuation system 17 that moves the piston rod 3 backwards as soon as the input effort F1 becomes less than or equal to the threshold F1ad. The retraction threshold F1ad is adjustable depending on the type of injection carried out by the user. When the injection requires great precision, the F1ad threshold can be raised near the trigger threshold of assistance F1s. In this case, a slight relaxation of the user makes it possible to stop the injection and to activate the return backward. On the other hand, when less precision is required, the F1ad threshold can be lower, and even zero. In this case, stopping the injection and activating the return backward are only effective when the user completely relaxes pressure exerted on the sensor.

In order to avoid any inadvertent backward return, a time value is recorded in the memory so that the return backward is only performed when the time interval Δt elapsed after the end of the injection is equal to this time value. The processor is advantageously provided with a clock programmed to measure the time interval Δt.

FIG. 9D shows a third example of a control law for anti-drip processes. (a) The output effort F2 remains zero until the input effort F1 exerted by the user on the sensor(s) 16a, 16b, 16c reaches the trigger threshold F1s after which the assistance by control law is triggered; (b) the output effort F2 increases as the injection proceeds; (c) the output effort F2 decreases with the decrease in input effort F1 after injection until it reaches zero (returns backward along the curve); (d) the output effort F2 continues to decrease and passes below the zero starting value when F1 reaches the value F1ad, which results in a return of the piston rod in the proximal direction by an adjustable predetermined distance, and the aspiration of the drop.

The intake effort F1 is not completely zero, because the user still has the device in hand and therefore is exerting an effort, although weak, on the sensor. As long as the input effort is between F1ad and F1s, assistance remains deactivated.

### Detection of Blood

During injection of a composition into irrigated tissues, the needle could puncture a blood vessel. Injection devices of the state-of-the-art offer no possibility of detecting the penetration of a blood vessel by the needle.

In order to overcome this disadvantage, according to a fourth example, the control law comprises a process that makes it possible to carry out a calibrated aspiration in order to detect the aspiration of blood by the end of the needle that flows back to the proximal end of the needle. If the user determines that no blood has been aspirated, he can continue the injection. Otherwise, he moves the needle in the tissues. The triggering of the blood detection procedure by the user can be done by a dedicated pushbutton or by using time information from one of the force sensors, for example by pressing twice quickly.

FIG. 9E shows a fourth and fifth control law example. These examples represent the fact that the activation threshold F1s is adjustable (value F1s' or F1s″), particularly based on the syringe and the use of the injection device. Although this is not shown, the limit F1I, the limit F2max, as well as the threshold F1 are also adjustable.

FIG. 10 is a graph that shows the evolution of the axial position Z of the piston rod along the injection axis of the device as a function of time t, during a series of injections of the same composition.

(a) The device is placed in operation. The piston rod 3 seeks its starting position and is therefore moved distally until coming into contact with the proximal stop element 27. In the graph, this is represented by a decrease of Z as a function of t. The electromechanical system then launches an automated step of approaching the piston 50 by the piston rod 3. The piston rod is therefore moved distally automatically until it is pressed against the piston. In the graph, this is represented by an increase in Z.

(b) The device then performs a process of detecting a blood vessel when triggered by the user. The calibrated aspiration and the rearward movement of the piston rod 3 results in a small decrease in Z. The curve is then flat during a specific period of time, which corresponds to a waiting phase during which no effort is exerted on the sensor 16a, 16b, 16c.

(c) The injection is then carried out. The piston 50 is moved distally following the effort exerted by the user on the sensor, and a corresponding dose of composition is injected. Z increases then as a function of t.

(d) The device then performs an anti-drip process. The backward return of the piston rod 3 results in a small decrease in Z.

The series of injections is then continued, each injection (c′), (c″), etc., preferably being followed by an anti-drip process (d′), (d″), etc., and a waiting phase.

(e) At the end-of-travel of the piston rod, the device automatically starts a backward return step of the piston rod 3 of separating the piston rod from the piston 50. The piston rod is therefore moved automatically without intervention from the user on the piston rod, in the proximal direction until the gripping zone of the piston rod comes into contact with the proximal stop element 27.

1. An assisted manual injection device for injecting a composition contained in a container, wherein the container is provided with a needle at its distal end, and contains a piston, the injection device comprising:
a body comprising at least one gripping zone by which the body can be held in a user's hand,
a holding element of the container, configured to receive and hold the container in a stationary manner relative to the body,
an electromechanical system comprising:
a piston rod capable of being moved in translation in the container relative to the body of the device in order to move the piston,
an actuation system of the piston rod,
a processor capable of communicating with the actuation system,
wherein the device comprises at least one force sensor positioned on the body at a gripping zone, the force sensor being configured to receive, as input, a mechanical effort F1 exerted on said sensor by the user during the injection of the composition, and to transmit to the processor an output signal F2 that depends on the mechanical force,
and the processor is configured to send an instruction to the actuation system depending on the output signal from the sensor in order to actuate the piston rod depending on a predetermined control law, in order to adjust the mechanical effort that needs to be applied by the user to the piston rod in order to inject the composition.

2. The device according to claim 1, wherein the actuation system comprises an effort multiplication system configured to furnish to the piston rod a mechanical effort F2 which is a multiple of the effort F1 exerted by the user on the sensor, and wherein the control law is an effort multiplication law.

3. The device according to claim 1, wherein the control law defines a movement of the piston rod in a proximal direction along a predetermined distance D1 when the injection is interrupted and the mechanical effort F1 exerted by the user on the sensor is less than a threshold value F1ad determined during a specific duration Δt1.

4. The device according to claim 1, wherein the piston rod has a starting position, and the control law defines a movement of the piston rod in a proximal direction along a predetermined distance D2 when the injection is terminated and the mechanical effort F1 exerted by the user on the sensor is less than a threshold value F1ad determined during a specific duration Δt2 or when the piston rod is at the distal end of travel.

5. The device according to claim 1, wherein the needle being positioned in a subcutaneous zone of the human body, the control law defines a movement of the piston rod in a proximal direction according to a predetermined distance D3 in order to perform a calibrated aspiration of a specific volume of a human fluid, in order to detect the possible presence of blood in the needle.

6. The device according to claim 1, comprising a selective activation means allowing the user to selectively activate or deactivate the control of the piston rod by the processor via the control law.

7. The device according to claim 6, wherein the selective activation means comprises an adjustment means allowing the user to choose a trigger threshold F1s, in such a way that the control of the piston rod by the processor via the control law is triggered only when the effort F1 exerted by the user on the sensor is greater than or equal to the trigger threshold F1s.

8. The device according to claim 1, wherein the body of the injection device comprises at least two gripping zones on a distal surface of the body of the device, where at least one of the gripping zones is provided with a sensor and is able to receive pressure from the index finger, the middle finger, the ring finger or the little finger of the user, and the piston rod comprises a proximal end comprising a gripping zone, said gripping zone being provided with a sensor and being able to receive the pressure from the user's thumb to allow the device to be held like a syringe.

9. The device according to claim 8, wherein the gripping zone of the piston rod is positioned on a trigger integral with the piston rod and which extends obliquely or perpendicular to the piston rod from a distal position relative to the proximal end of said piston rod.

10. The device according to claim 1, comprising at least three gripping zones arranged on the body of the device around a longitudinal axis of the device, at least one of said gripping zones being provided with a sensor said gripping zones being able to receive respectively the pressure from the user's index finger, middle finger, or thumb in order to allow the device to be held like a pen.

11. The device according to claim 1, wherein the holding element of the container comprises a pivotable cover provided with an orifice capable of receiving at least the proximal end of the container, the pivotable cover being capable of pivoting between an injection position in which the orifice is aligned with the travel path of the piston rod, and a free position in which the orifice is offset relative to the travel path of the piston rod and is accessible by the user to allow the insertion of the container into said orifice.

12. The device according to claim 1, wherein the holding element of the container further comprises a plurality of interchangeable rings provided with orifices of different diameters adapted to receive corresponding containers of different diameters.

13. The device according to claim 12, wherein the pivotable cover is produced in several parts, of which at least a first part is integral with the body of the device, and at least a second movable part capable of pivoting between the injection position and the free position, the two parts of the pivotable cover thus forming a clamp of variable spacing making it possible to receive and clamp the container in order to maintain it in a stationary position.

14. The device according to claim 13, wherein the second movable part is capable of being detached from the body of the device.

15. The device according to claim 14, wherein the second movable part is color-coded to be identifiable by the user or is provided with a recognition and electronic reading device in order to specify to the processor the geometric or rheological characteristics of the container and its contents so that the recognition device can automatically calculate the dose of composition delivered for a unitary movement of the piston rod.

16. The device according to claim 1, wherein the electromechanical system comprises a memory in which control laws and/or threshold values are recorded.

17. The device according to claim 1, further comprising a user interface allowing the user to record and select the operating parameters of the device, and to have information feedback from the device about said operating parameters during the use of the device.
